(19) United States
(12) Patent Application Publication
(10) Pub. No.: US 2002/0002327 A1
(43) Pub. Date: Jan. 3, 2002

(54) Method for detecting cheyne-stokes respiration in patients with congestive heart failure

(76) Inventors: Brydon J.B. Grant, East Amherst, NY (US); Ali El-Solh, West Seneca, NY (US)

Correspondence Address: Ranjana Kadle, Hodgson Russ LLP, One M&T Plaza, Suite 2000, Buffalo, NY, 14203-2391, US

(21) Appl. No.: 09/829,695

(22) Filed: Apr. 10, 2001

Related U.S. Application Data
(60) Provisional application No. 60/195,804, filed on Apr. 10, 2000.

(51) Int. Cl. A61B 5/00

(52) U.S. Cl. 600/324; 600/529

(57) ABSTRACT

The present invention discloses a method for developing a diagnostic method for CSR. The method comprises the steps of performing overnight oximetry recordings in patients suspected of OSA who have been identified as having or not having CSR by clinical studies. Spectral analysis is performed on the oximetry recordings from which a classification tree is generated. The present invention also discloses a method for the identification of Cheyne-Stokes respiration in an individual. The method comprises the steps of performing spectral analysis of overnight oximetry recordings. The key features are then input into a classification tree to determine the presence or absence of CSR.

This application claims the priority of U.S. provisional application serial No. 60/195,804 filed on Apr. 10, 2000, the disclosure of which is incorporated herein by reference.

### FIELD OF THE INVENTION

The present invention relates generally to the field of sleeping disordered breathing. More particularly, the present invention provides a diagnostic method for the detection of Cheyne-Stokes respiration (CSR), and a method for developing such a diagnostic method.

### DISCUSSION OF RELATED ART

Sleep disordered breathing (SDB) is estimated to occur in about 60% of patients suffering from congestive heart failure (CHF; Rechtschaffen A, Kales A, eds. A Manual of Standardized Technology, Techniques and Scoring System for Sleep Stages of Human Subjects. Los Angeles: UCLA Brain Information Service/Brain Research Institute, 1968). Cheyne-Stokes respiration (CSR) is by far the most common form of SDB encountered with an estimated prevalence of 40% (Javaheri et al., 1995, Ann Intern Med., 122:487-92; Findley et al., 1985, South Med. J., 78:11-5). It is characterized by rhythmic rises and falls in tidal volume and breathing frequency that lead to oxygen desaturation, increased arousals, poor sleep quality, and altered sleep architecture. These features result in complaints of daytime somnolence, fatigue, and insomnia.

The pathophysiology of CSR is not completely understood, but it has become more apparent that the effect of altered breathing patterns may extend beyond the deterioration in psycho-cognitive function. The increase in urinary and plasma norepinephrine levels in patients with left ventricular failure (LVF) and CSR compared to those with CSR alone has been implicated in an accelerated loss of cardiac function, and an increased risk of death and cardiac transplantation (Naughton et al., 1995, Am J. Respir Crit Care Med, 152:473-79; Hanly et al., 1996, Am J Respir Crit Care Med, 153:272-76). Nasal continuous positive airway pressure (CPAP) has been advocated as an effective nonpharmacological treatment for patients with congestive heart failure and CSR. Recent studies have shown that CPAP can abolish CSR, improve respiratory muscle strength (Granton et al., 1996, Am J Respir Crit Care Med, 153:277-82), and increase left ventricular ejection fraction (Naughton et al., 1993, Am Rev Respir Dis, 148:330-38), and may increase transplant-free survival.

In the absence of a good and accurate screening test, overnight polysomnography remains the gold standard test for the diagnosis of CSR. However, overnight polysomnography is an expensive, labor intensive and time-consuming procedure. Home pulse oximetry has been proposed as an alternative tool for identification CSR, but relies on visual inspection of the oximetry signal by a trained observer (Staniforth et al., 1998, Heart, 79:394-99).

The presence of CSR has been implicated in the increased mortality up to 56% over a 3 year-period compared to 11% in those patients without CSR despite similar cardiac functional status and left ventricular function (Hanly et al., 1996, supra). Since nasal CPAP therapy was found to have a beneficial acute and chronic cardiovascular effect, early implementation might well be translated into improved cardiac function, reduced hospitalization and potentially reduced mortality. Thus, there is an ongoing need for more accurate methods in the detection of Cheyne-Stokes respiration.

### SUMMARY OF THE INVENTION

The present invention provides a diagnostic method for the identification of CSR, and a method for developing the diagnostic method.

The method for developing the diagnostic method comprises the steps of performing clinical studies on patients suspected of having obstructive sleep apnea. Based on the clinical studies, patients are identified as having or not having CSR. Overnight pulse oximetry recordings are obtained from these individuals following which spectral analysis is performed on the oximetry recordings. From the spectra, a set of parameters or key features are determined and used to build a classification tree that enables the prediction of CSR. The tree is tested by cross validation.

The diagnostic method for detecting the presence or absence of CSR in an individual comprises the steps of obtaining overnight oximetry recordings from the individual, performing spectral analysis of the recordings, obtaining a set of parameters or key features from the spectra and inputting the parameters into a classification tree.

### BRIEF DESCRIPTION OF THE DRAWINGS

FIG. 1 is a representation of the steps for developing a diagnostic method according to the present invention.

FIG. 2 is a representation of a power spectra of pulse oximetry in two representative patients, one with severe obstructive sleep apnea (OSA; AHI>40/hr) and another without OSA (AHI<5/hr). Magnitude is plotted on the ordinate against frequency on the abscissa. The continuous line is the spectrum of a patient with an apnea-hyponea index less than 5/h and the interrupted line is the spectrum of a patient with an apnea-hyponea index greater than 40/h.

FIG. 3 is a representation of a power spectrum of pulse oximetry in a representative patient with Cheynes-Stokes respiration. Magnitude is plotted on the ordinate against frequency on the abscissa. The ordinate is expanded seven fold compared with FIG. 2.

FIG. 4 is a representation of a classification tree to identify patients with Cheyne Stokes respiration (CSR) from the characteristics of the power spectrum of pulse oximetry. M1 and M2 are the magnitudes of the highest and next highest local maximum normalized by the overall variance, m1 is the magnitude of the highest local maximum in absolute terms, n1 is the number of CSR patients and n2 is the number of non CSR patients in a category.

FIG. 5 is a representation of the receiver operator characteristic curve indicating the diagnostic accuracy of the regression tree for identifying patients with Cheyne Stokes respiration from patients suspected of obstructive sleep apnea. Sensitivity is plotted on the ordinate against (1-specificity) on the abscissa.

FIG. 6 is a representation of the steps for the diagnosis of CSR in an individual according to the present invention.

### DETAILED DESCRIPTION OF THE INVENTION

The present invention provides a method for developing a classification tree that can used to identify CSR and a method for using the classification tree to diagnose the presence or absence of CSR in an individual. The method is based on the observation that when oxygen saturation levels over selected time intervals are transformed to frequency distribution spectra, the spectral indices for those patients with CSR display characteristic features with distinctive discriminative attributes compared to other sleep disordered breathing. While the power frequency distribution (a plot of variance versus frequency) of normal subjects was shown to have no apparent peak, and of OSA patients to have broad-band peaks, the patients with congestive heart failure having CSR often had a unique distribution of spectral peaks conforming to a long-period oscillating output.

For developing the classification tree, individuals with suspected sleep apnea are identified from clinical sleep studies. Overnight oximetry recordings are obtained from individuals suspected of having OSA. Power spectra are generated from the oximetry recordings. A set of key features or parameters are obtained from the power spectra. These parameters are then used as input data to construct a classification tree.

The present invention also provides a diagnostic method for identification of CSR. The method of the diagnostic method comprises performing spectral analysis of overnight pulse oximetry data. The spectral data is then analyzed using a classification tree to obtain a predictive value that is indicative of the likelihood that an individual has CSR.

The present invention is also directed to a storage device, such as a floppy disk or hard drive, having thereon computer readable code for causing a computer to execute all or a substantial portion of diagnostic method.

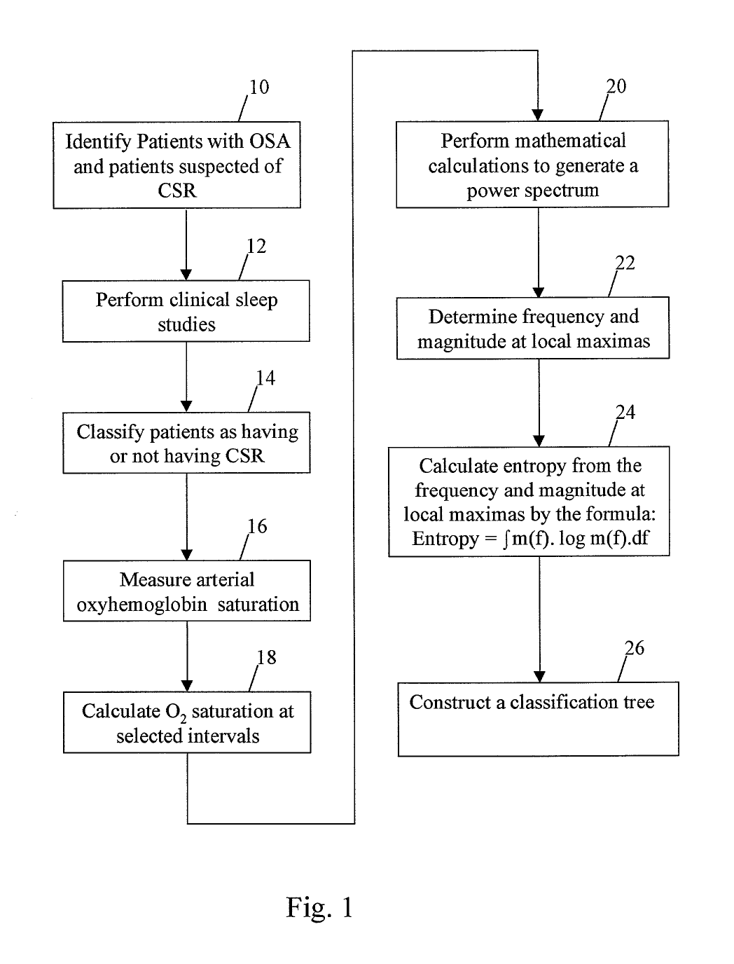

In one embodiment of the invention, a method for developing the diagnostic method is illustrated by the steps shown in FIG. 1 and is also illustrated by way of an example described below to construct a classification tree.

For developing a classification tree, patients suspected of obstructive sleep apnea were identified (Step 10). An analysis of sleep studies (Step 12) was performed in 248 patients at the Sleep Laboratory at the Veterans Affairs (VA) Medical Center in Buffalo, N.Y. (n=45) and at the National Sleep Technologies Laboratory in Syracuse, N.Y. Patients with left ventricular failure had been studied in the sleep laboratory in Buffalo as part on another study on sleep disordered breathing in patients with left venticular failure. All patients in Syracuse sleep laboratory were suspected of obstructive sleep apnea syndrome.

All the sleep studies were performed between February 1998 and June of 1999. Continuous electroencephalogram, electrooculogram, electrocardiogram, and submental electromyogram were recorded on a 16-channel polygraph using standard techniques, and digitized on a computerized system. The sleep data collection system was Aquetron 1000P at the Buffalo Va. and Healthdyne in Syracuse (Healthdyne 930, Pittsburgh, Pa.). Airflow was measured qualitatively by the sum of an oral-nasal thermistor (Graphic Control; Buffalo, N.Y.). Thoracoabdominal movements were recorded with an inductive plethysmograph in Buffalo (Respitrace, Ambulatory Monitoring, Ardsley, N.Y.) and with peizoelectric method in Syracuse.

Sleep stages were scored in 30-sec epochs using the Rechtschaffen and Kales sleep scoring criteria (1968, A Manual of Standardized Technology, Techniques and Scoring System for Sleep Stages of Human Subjects. Los Angeles: UCLA Brain Information Service/Brain Research Institute). Each epoch was analyzed for the number of apneas, hypopneas, arousals, oxyhemoglobin desaturation, and disturbances in cardiac rate and rhythm. Apnea was defined as the absence of airflow for more than 10 seconds. Hypopnea was defined as a visible 20% reduction in the airflow lasting more than 10 seconds associated with either 4% oxygen decrease in arterial oxyhemoglobin saturation or an electroencephalographic arousal, or both. Central apneas were defined by the cessation of airflow for 10 seconds accompanied by an absence of chest wall movement. The apnea-hypopnea index (AHI) was defined as the number of apneas and hypopneas per hour of sleep. The presence of CSR was defined as a central apnea index of ≧≧5 per hour of sleep, in combination with the characteristic pattern of crescendo-decrescendo pattern of hyperpnea alternating with hypopneas. An arousal was defined as recommended by the American Sleep Disorders Association's position paper as a change in electroencephalogram rhythmn for greater than 3 sec. (Guilleminault et al., 1992, Sleep, 15:173-84).

The frequency spectra of SpO₂ from the 23 patients with CSR was compared with the spectra of 203 patients suspected of obstructive sleep apnea, and a validated model to identify the patients with CSR was developed. The model was tested by determining its specificity in patients with left ventricular failure who did not have CSR (n=22).

Gated ⁹⁹Tc equilibrium radionuclide angiography obtained within 6 month of the diagnostic sleep study was used as an objective measurement of cardiac function in those with documented CSR on overnight polysomnography. The quantitation and reporting of left ventricular function were preformed by trained technicians and a nuclear medicine physician blinded to the patient's sleep study findings.

Of the 248 patients, 221 (89%) were men and 26 (11%) were female. Forty four patients had congestive heart failure with a mean left ventricular ejection fraction (LVEF) of 24.9±9.1%. The largest proportion of patients was in NYHA class 2 (57%). Fifteen patients were in NYHA class 3 (34%), while 4 (9%) were in NYHA class 4. The causes of LVF were attributed to ischemic heart disease in 82% of the cases, nonischemic dilated cardiomyopathy in 16%, and others in 2%. Baseline age, body mass index, and LVEF were similar between those who met the criteria for central sleep apnea and those who did not (Table 1).

TABLE 1
Characteristics of left ventricular failure
patients with and without central sleep apnea.

| | Central sleep | No central sleep |
|---|---|---|
| | apnea | apnea |
| | (n = 22) | (n = 22) |
| Age (years) | 71 ± 4 | 68 ± 9 |
| Body mass | 24.2 ± 3.8 | 26.9 ± 4.1 |
| index* (kg/m²) | | |
| Left | 23 ± 5.7 | 27 ± 6.2 |
| ventricular | | |
| ejection | | |
| fraction (%) | | |

*Body mass index is the weight in kilograms divided by the square of the height in meters.

Table 2 lists the characteristics of sleep and disordered breathing events and oxyhemoglobin saturation during sleep in those patients.

TABLE 2
Sleep studies characteristics of patients with
left ventricular failure

| | Central | No Central | |
|---|---|---|---|
| | Apnea | Apnea | |
| | (n = 22) | (n = 22) | p value |
| Total recording | 417 ± 48 | 453 ± 62 | 0.6 |
| time (min) | | | |
| Total sleeping | 282 ± 98.5 | 307 ± 54 | 0.4 |
| time (min) | | | |
| Sleep efficiency | 66 ± 19 | 68 ± 32 | 0.7 |
| (%) | | | |
| Arousal index (/h) | 23 ± 19 | 12 ± 8 | 0.04 |
| Apnea-hypopnea | 32 ± 13 | 3.6 ± 2 | <0.01 |
| index (/h) | | | |
| Central apnea | 22.7 ± 14.6 | 0.7 ± 1.6 | <0.01 |
| index (/h) | | | |
| SpO₂ baseline | 92 ± 2 | 94 ± 4 | 0.4 |
| value (%) | | | |
| SpO₂ lowest value | 77 ± 9 | 89 ± 2 | <0.01 |
| (%) | | | |
| % time SpO₂ < 90% | 34 ± 31 | 14 ± 6 | <0.01 |

SpO₂ is the oxygen saturation by pulse oximetry

Among patients with CSR, the mean central sleep index was 22.7±14.6. Arousal index was significantly higher, and arterial oxyhemoglobin desaturation was significantly lower in CSA patients compared to those without CSA, but the differences in total sleeping time, and sleeping efficiency were not statistically significant.

Of the 203 remaining patients referred for evaluation of sleep disorders, 152 had polysomnographic evidence of obstructive sleep apnea (OSA). Thirty seven (18%) had severe OSA with AHI >40/hr, 47 (23%) had moderate OSA with AHI ranging between 20 and 40/hr, and 68 (33%) had mild OSA with AHI between 5 and 20/hr.

Based on the clinical studies, individuals were classified as having or not having CSR (step 14). In the next step 16, measurement of arterial oxyhemoglobin saturation was performed with a pulse oximeter with the probe placed on the patient's finger. In Syracuse, oximetry data were recorded with two seconds sampling interval with the oximetry sampling rate of 300 Hz and the data smoothed with a moving average of 4 seconds. In Buffalo, the oximetry (Ohmeda 3720, Louisville, Colo.) data was sampled at 400 Hz and the data smoothed with a moving average of 3 sec.

The raw data was processed to remove any artifacts by eliminating all changes of oxygen saturation between consecutive sampling intervals of greater than 4% per second, and any oxygen saturation less than 20%. The lowest value of the oxygen saturation by pulse oximetry (SpO₂) over 4 seconds intervals was determined (Step 18) and used for spectral analysis. Only the longest section of data free of artifacts on each subject was used for spectral analysis. In the next step 20, a power spectral was generated using the maximum entropy method. This approach is well known to those skilled in the art. It differs from Fourier transform methods and is explained in detail in Press et al. (1989, Numerical recipes New York, Cambridge University Press Chapter 12, Fourier transform spectral methods, 381-453). The power spectrum provides a measure of the variability of oxygen saturation that occurs over a range of frequencies. The magnitude of that power is related to the variance (square of the standard deviation). To determine optimal model size that minimizes the tradeoff between increased accuracy and increasing the variance of the estimated spectrum, the Bayesian information criterion was used. (Hurvich et al., 1989, Biometrika, 76:297-307).

The next step (step 22) was to determine a set of parameters from the power spectra. The spectrum covered frequencies between 0.00125 and 0.125 Hz. The key features of the power spectrum that identified to characterize the spectra of CSRs were the frequency and the magnitude of the power attained at the highest local maximum (f1, m1), and the frequency and the magnitude of the power attained at the next highest local maximum (f2, m2). A local maxima of magnitude in the spectrum was identified when there were lower magnitudes at frequencies immediately above and below the particular frequency. The spectrum generated between 0.00125 and 0.125 Hz at 100 frequencies equispaced on a log scale. The absolute magnitude (m1 and m2) were also normalized by the variance (M1 and M2) and ther values incorporated into the model. The spectra were also characterized by the amount of entropy (randomness) in the data.

In the next step (Step 24) the entropy was measured by

Entropy=−∫(m(f)*log m(f).df

where ∫ is the summation of the magnitudes of the spectrum at equidistant intervals of frequency on a linear scale between 0.00005 and 0.05 Hz, and m(f) represents the magnitude at specific frequency f. Heuristically, the entropy has been interpreted as a measure of uncertainty about the event f. High uncertainty (entropy) is due to a large number of processes, whereas low entropy is due to a small number of dominating processes which make up the time series.

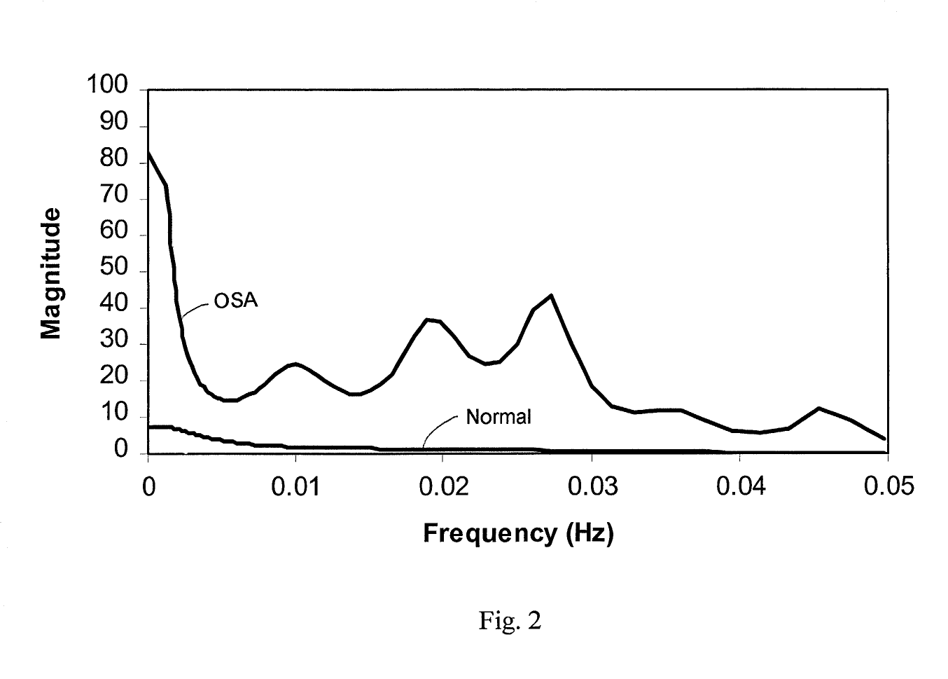

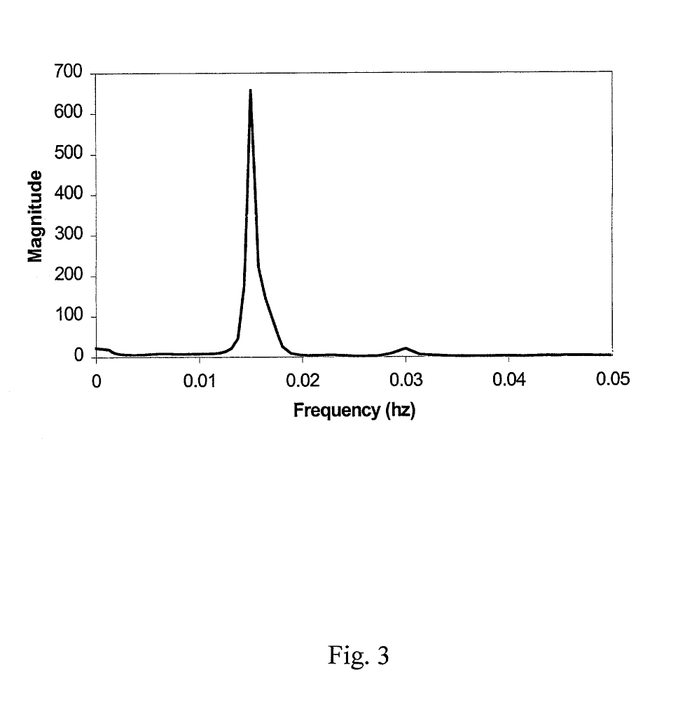

Representative examples of the power spectra a normal individual, a patient with with OSA, and a patient with CSR are displayed in FIGS. 2 and 3. The power spectrum in CSR patients is characterized by a sharp spectral peak with a large primary local maximum displayed at low frequency (<0.02 Hz). In contrast, the power spectrum in OSA consists of multiple, broad-band spectral peaks, lower in magnitude with the highest local maximum located at a frequency ≧0.02 Hz. In normal subjects, no apparent peak was detected. Table 3 shows the values (mean ± SD) of the various indices of the spectral analysis in CSR, OSA patients, and normal controls.

TABLE 3
Summary of the results of spectral analysis

| | | | Suspected |
|---|---|---|---|
| | LVF-CSR | LVF-No CSR | OSA |
| | n = 23 | n = 22 | n = 203 |
| Magnitude at | 19 (19.8) | 3.1 (4.6) | 2.18 (0.3) |
| primary local | | | |
| maximum (m1) | | | |
| Magnitude at | 18 (18.9) | 3.1 (4.6) | 1.88 (0.29) |
| secondary local | | | |
| maximum (m2) | | | |
| Variance | 8.39 (7.97) | 4.5 (6.8) | 4.82 (7.26) |
| Entropy | 4.44 (0.77) | 4.3 (0.74) | 5.24 (0.77) |

*The results are expressed in terms of means ± SD. LVF is left ventricular failure and CSR is Cheyne Stokes respiration.

In the next step 26, a classification tree was developed with binary recursive partitioning to identify patients with CSR according to the method of Breiman et al. (1984, Classification and Regression Trees, Belmont, Calif., Wadsworth International Group). In brief, the input data consisted of magnitude and frequency values. The output variable was coded as 1 for the presence of CSR and 0 for the absence of CSR. Because of the preponderance of patients with suspected obstructive sleep apnea, the patients with CSR were weighted by a factor of 10.

The root of the tree is determined by the probability of CSR based on the prevalence in the data set. Next, each variable is selected in turn to determine the most accurate predictor of CSR. The data at the first node is then separated into two branches. At the end of each branch, a new node is developed and the input variables are retested to determine which one produces the most accurate classification into those with CSR and those without. The optimal size of the tree was found by five-fold cross validation.

A receiver operator characteristic (ROC) curve was generated to assess the accuracy of the regression tree. The c-index, which is equivalent to the area under the curve, was used to estimate the diagnostic accuracy of the model. The c-index and its standard error were calculated by the bootstrap method that has been described previously (El-Solh et al., 1996, Chest, 110:1299-1304).

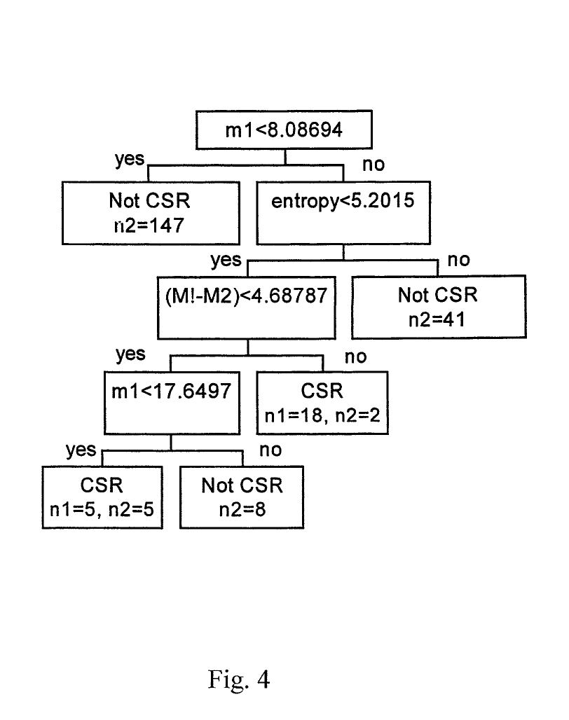

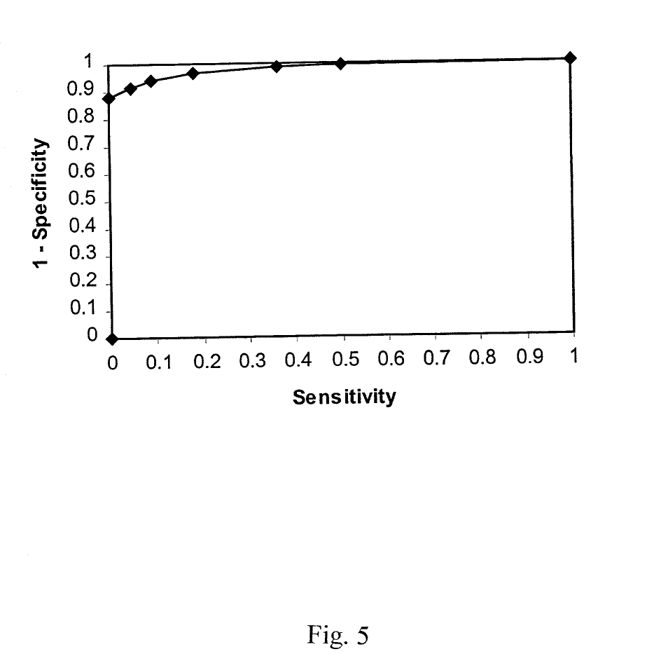

An example of the classification tree is presented in FIG. 4. The tree was grown by binary recursive partitioning and was shrunk to determine its optimal size using tenfold cross-validation. It was pruned accordingly to avoid overfitting. The tree predicted that CSR was unlikely to be present if the magnitude of the power (m1) at the highest local maximum was less than 8.0867 (%). For those with a local maximum greater than 8.0867, an entropy greater than 5.202 is unlikely to indicate CSR. Of those with a lower entropy, CSR is likely to be present if the difference in the normalized magnitudes between the highest and next highest local maxima was greater than 4.688. Otherwise, CSR will be present only in those with a highest local maximum less than 17.645. When tested on the entire data set, the tree achieved a sensitivity of 100% (95% CI 85%-100%) and a specificity of 97% (95% CI 93%-99%). Seven patients who did not have CSR were classified erroneously as having CSR by the regression tree. The accuracy of the regression tree was assessed with a ROC curve shown in FIG. 5. The c-index, which is equivalent to the area under the curve, was 0.997 (95% CI 0.992-1.0%).

All results are expressed as mean ± standard deviation. Differences between patients were compared by the Student's unpaired t test, and frequency events by chi-square test with Yates' correction. All tests of statistical significance were two sided. A p value of 0.05 was considered to be statistically significant. Commercially available software was used to develop the regression tree (S-Plus; Statsci; Seattle, Wash.), and for confidence interval (CI) analysis (CIA; British Medical Journal; London, England).

To determine the predictive value of the diagnostic method developed as described above, the classification tree constructed as in FIG. 4 was tested on 22 patients with LVF who had no evidence of CSR by overnight polysomnography. Of these 22 patients, two patients were mis-classified as having CSR yielding a specificity of 91 (95% CI:71-99%) and the positive and negative predictive ratios were 92% (95% CI 74-95%) and 100%( 95% CI: 83-100%).

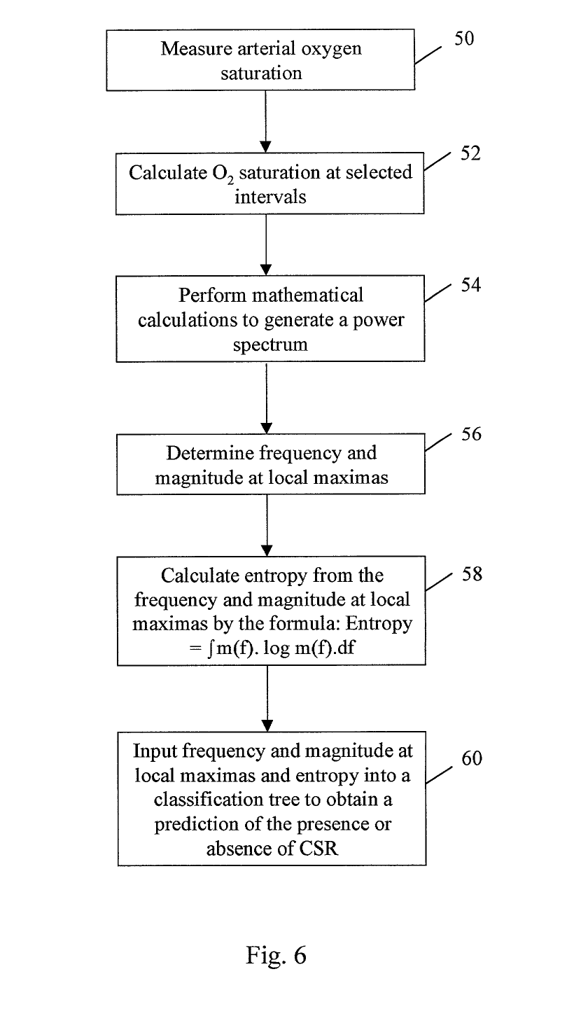

In another embodiment of the invention, the classification tree developed as described herein is used in a diagnostic method to identify CSR in an individual. The diagnostic method comprises the steps shown in FIG. 6. Blood oxyhemoglobin saturation levels are obtained from a patient by pulse oximetry recordings (Step 50). Oxygen saturation levels are determined at selected intervals (Step 52). Mathematical calculations are performed to generate a power spectrum (Step 54) from the pulse oximetry readings by plotting magnitude (variance) versus frequency. From the power spectrum, a set of parameters of magnitude and frequency are attained at the highest local maximum (f1, m1) are determined (Step 56). Similarly, the frequency and magnitude of the power attained at the next highest local maxima (f2, m2) are determined. A local maxima of magnitude is identified when there is lower magnitudes at frequencies immediately above and below the particular frequency.

In the next step (step 58), entropy is calculated by the following formula:

Entropy=−∫(m(f)*log m(f).df

where ∫ is the summation of the magnitudes of the spectrum at equidistant intervals of frequency on a linear scale between 0.00005 and 0.05 Hz, and m(f) represents the magnitude at specific frequency f.

In the next step (step 60), the set of parameters and the entropy value determined are input into a classification tree developed as described herein to obtain a prediction of whether the individual has CSR or not.

From the foregoing, it will be obvious to those skilled in the art that various modifications in the methods described herein can be made without departing from the spirit and scope of the invention. Accordingly, the invention may be embodied in other specific forms without departing from the essential characteristics thereof. The embodiments and examples presented herein are therefore to be considered as illustrative and not restrictive.

What is claimed is:

1. A method for detecting Cheyne-Stokes respiration in an individual comprising:
obtaining pulse oximetry recordings from the individual;
determining oxygen saturation levels at selected intervals;
generating a power spectrum from the oxygen saturation levels;
determining a set of parameters from the power spectrum comprising frequency and magnitude at local maximas; and
inputting the set of parameters into a classification tree to determine the presence or absence of CSR.

2. The method of claim 1, wherein the pulse oximetry readings are obtained overnight.

3. A storage device having stored thereon computer readable code for causing a computer to execute the method of claim 1.

4. A method for developing a classification tree for identification of individuals with CSR comprising the steps of:
performing clinical studies to identify patients having obstructive sleep apnea;
performing further clinical studies on patients identified as having obstructive sleep apnea to determine the presence or absence of Cheyne-Stokes respiration;
obtaining overnight pulse oximetry recordings from the patients with or without Cheyne-Stokes respiration;
determining oxygen saturation levels at selected intervals;
generating power spectra from the oxygen saturation levels;
determining a set of parameters from the power spectra comprising frequency and magnitude of local maximas; and
constructing a classification tree by inputting the set of parameters.

5. The method of claim 4 further comprising the step of cross-validating the classification tree.

6. The method of claim 4, wherein the further clinical studies are other than pulse oximetry recordings.
